(19) United States
(12) Patent Application Publication
(10) Pub. No.: US 2023/0235371 A1
(43) Pub. Date: Jul. 27, 2023

(54) METHOD FOR CULTURING MICROORGANISMS FOR LIPID ACCUMULATION

(71) Applicant: FERMENTALG, Libourne (FR)

(72) Inventors: Said IHAMMOUINE, Libourne (FR); Francois GODART, Vayres (FR)

(21) Appl. No.: 18/002,357

(22) PCT Filed: Jun. 24, 2021
(86) PCT No.: PCT/EP2021/067279

§ 371 Date: Dec. 19, 2022

(30) Foreign Application Priority Data
Jun. 24, 2020 (FR) FR2006599

(51) Int. Cl. C12P 7/6472; C12N 1/14

(52) U.S. Cl. CPC C12P 7/6472; C12N 1/14

(57) ABSTRACT

The present invention relates to the industrial cultivation of protists for producing lipids containing polyunsaturated fatty acids (PUFA), in particular eicosapentaenoic acid (EPA) and arachidonic acid (ARA).

### FIELD OF THE INVENTION

The present invention relates to the industrial cultivation of protists for producing lipids containing polyunsaturated fatty acids (PUFAs), in particular eicosapentaenoic acid (EPA) and arachidonic acid (ARA).

### STATE OF THE ART

Protists of the genus Pythium are filamentous microorganisms belonging to the class of Oomycetes having similarities with fungi. Pythium has the particularity of synthesizing and accumulating reserve lipids containing Polyunsaturated Fatty Acids (PUFA) such as Eicosapentaenoic Acid (EPA) and Arachidonic Acid (ARA). The interest of this protist lies in the majority accumulation of EPA among the other PUFAs, in particular ARA.

Some have therefore considered using Pythium as an industrial strain for producing oil rich in EPA (EP 1 001 034; WO 2014/137894; Lio J, Wang T. (2013); Liang Y, Zhao X, Strait M, Wen Z. (2012); Athalye SK, Garcia RA, Wen Z. (2009); Stinson EE, Kwoczak R, Kurantz MJ. (1991); Gandhi SR, Weete JD. (1991)).

Pythium is a non-photosynthetic microorganism which develops in an aqueous medium or in the soil, often as a parasite on wild or cultivated plants (cereals and vegetable crops). The cultivation of Pythium is carried out in heterotrophy, that is to say in the absence of light. This is also one of the advantages identified in the state of the art of not having to depend on light (WO 2009/143007).

However, the fat content in the strains cultivated by these cultivation methods, less than 30% of the dry matter, remains too low for industrial exploitation.

The present invention allows to overcome this problem with a new cultivation method which allows to increase the fat content to levels of percentages of dry matter compatible with industrial exploitation.

### DISCLOSURE OF THE INVENTION

The present invention relates to a method for preparing a biomass of protists of the genus Pythium comprising PUFA-rich lipids, said method comprising (a) the cultivation of protists of the genus Pythium on a cultivation medium comprising a carbon source, and (b) the recovery of the biomass from the cultivation medium, the cultivation step comprising an illumination phase.

The invention also relates to a method for preparing lipid compositions rich in PUFA which comprises the cultivation from a) the cultivation of protists of the genus Pythium on a cultivation medium comprising a carbon source, (b) the recovery of the biomass from the cultivation medium and (c) the extraction of the PUFA-rich lipids from the recovered biomass, the cultivation step (a) comprising an illumination phase.

The invention also relates to a method for preparing a pharmaceutical, cosmetic, nutraceutical or food composition comprising a biomass of protists of the genus Pythium comprising PUFA-rich lipids, said method comprising (a) the cultivation of protists of the genus Pythium on a cultivation medium comprising a carbon source, (b) the recovery of the biomass from the cultivation medium and (d) the formulation of a composition by adding the biomass recovered in (b) to usual components of the pharmaceutical, cosmetic, nutraceutical or food compositions, the cultivation step (a) comprising an illumination phase.

The invention also relates to a method for preparing a pharmaceutical, cosmetic, nutraceutical or food composition comprising a lipid composition rich in PUFA, said method comprising (a) the cultivation of protists of the genus Pythium on a cultivation medium comprising a carbon source, (b) the recovery of biomass from the cultivation medium, (c) the extraction of the PUFA-rich lipids from the recovered biomass, and (d) the formulation of a composition by adding the lipid composition extracted in (c) to usual components of the pharmaceutical, cosmetic, nutraceutical or food compositions, the cultivation step (a) comprising an illumination phase.

The invention also relates to a biomass of protists of the genus Pythium comprising PUFA-rich lipids, the lipid content being at least 30% relative to the dry matter (% DM), preferably at least 50% DM, more preferentially, at least 55% DM, in particular at least 56, 57, 58, 59, 60, 61, 62, 63, 64 or 65% DM.

The protists of the genus Pythium are advantageously protists of the species Pythium irregulare.

### DETAILED DESCRIPTION OF THE INVENTION

### I. Definitions

“Biomass” means a set of cells of protists of the genus Pythium produced by their cultivation and separated from the cultivation medium (also called fermentation juice). The cells may or may not have retained their physical integrity. It is therefore understood that said biomass may comprise an amount of degraded protist cells ranging from 0% to 100%. “Degraded” means that the physical integrity of said cells of microorganisms may have been altered, such as lysed microorganisms, resulting for example from a homogenization or enzymatic lysis method. Once produced, this biomass may be raw, just separated from its cultivation medium, dried or not, degraded or not.

A raw biomass extracted from the cultivation medium can have a moisture content of 70% to 90%, generally 80% to 85%.

Dried biomass or “dry biomass” has a moisture content of 1% to 10%, generally 2% to 7%.

“Protists of the genus Pythium” means all the protists designated under the class of Oomycetes and of the Genus Pythium, producer of EPA, and capable of being cultivated industrially. This also includes strains with improved performance obtained by mutagenesis and selection, or by modification of the genome. This set of designated protists comprises in particular the species Pythium insidiuosum, Pythium irregulare, Pythium intermedium, Pythium splendens, Pythium ultimum. According to a preferred alternative of the invention, the protists of the genus Pythium are of the species Pythium irregulare.

“Industrial”, “industrial cultivation” or “industrially cultivated”, means a cultivation of the protists in a cultivation medium suitable for their growth and for producing PUFAs and in a volume suitable for producing the sufficient quantities to address a market. These industrial cultivations are carried out by fermentation in discontinuous mode called “batch”, in semi-continuous mode called “fed batch” or in continuous mode. Fermenters have volumes that can range from 1000 L to more than 200 m³.

“Cultivation medium” means an aqueous composition comprising the nutrients necessary for the growth of protists of the genus Pythium, in particular a source of carbon, a source of nitrogen, a source of phosphorus, but also mineral salts, and/or vitamins, trace elements, etc. well known to the person skilled in the art.

The cultivation medium can be a chemically defined medium, of known and reproducible composition, in which the content of each element is known and not comprising rich or complex organic and/or mineral matter.

The cultivation medium may alternatively comprise components of variable composition, such as rich or complex organic and/or mineral matter.

Rich or complex organic materials mean unpurified organic materials, in the form of mixtures for which the exact composition and the concentrations of the various components of the mixture are not known with accuracy, not mastered, and can have a significant variability from one batch to another. As an example of rich or complex organic material, mention may be made of yeast extracts or peptones which are products of a protein hydrolysis reaction or else plant infusions such as potatoes or rich mineral matter for example marine mineral salts or other complex growth agents, having no fixed concentration of each of their components.

The composition of the cultivation medium can vary over time with the consumption of nutrients by strains of protists of the genus Pythium. Depending on the cultivation method selected, it is possible to supplement the cultivation medium during the cultivation by feeding it with one or more complementary cultivation media which contain all or part of the nutrients present in the initial cultivation medium.

“Carbon source” means more particularly a complex carbon source which is not CO2, in particular selected from sugars, organic acids or polyols, such as glucose, cellulose derivatives, lactate, starch, lactose, sucrose, acetate, glycerol, fructose, xylose, any product or co-product rich in one or more of the aforementioned compounds and mixtures thereof.

“PUFAs”, abbreviation of “Poly Unsaturated Fatty Acid” are polyunsaturated fatty acids comprising at least 16 carbon atoms and at least 2 unsaturations, in particular the fatty acids identified by the signs ω3 and ω6, such as α-linolenic acid (ALA or C18:3n3), g-linolenic acid (AGA or (C18:3n3), arachidonic acid (ARA or C20:4n6), eicosapentaenoic acid (EPA or C20:5n3), docosahexaenoic acid (DHA or C22:6n3) or docosapentanoic acid (DPA or C22:5n6).

“PUFA-rich lipids” means a PUFA-rich oil comprising at least 10% of PUFA selected from EPA and ARA relative to the total mass of lipids, preferably at least 15%. The total mass of lipids represents at least 50% of the dry biomass, advantageously at least 60%, more advantageously at least 65%,

The PUFA-rich oils according to the invention are essentially in the form of triglycerides. The triglycerides represent at least 80% of the total fat mass, advantageously at least 90%, more advantageously at least 93% of the total fat mass. The triglyceride content is for example analyzed by thin layer chromatography (Jouet and al., 2003). These characteristics of the oil according to the invention relate both to the oil as present in the biomass of microorganisms and to the oil extracted from this biomass, whether crude or purified.

An oil called “crude” oil is an oil extracted from biomass after separation of lipids from the aqueous phase and insolubles, in particular proteins. The methods for extracting lipids from biomass are well known to the person skilled in the art, described in particular

WO 2020/053375, WO 2001/053512, WO 2011/153246, US 2014/350222, WO 2015/095694, WO 01/53512, WO 2010/096002.

An oil called “refined” oil is a purified oil obtained after purification of the crude oil, in particular refined according to its intended use. The methods for refining crude lipid compositions are well known to the person skilled in the art, described in particular by Manjula and al. (2006), GB 2 031 290, US 5 310 487 or US 4 971 660.

“Modified oil” means a crude or refined oil whose fatty acid composition has been modified. This modification can comprise a PUFA concentration, by eliminating other saturated or unsaturated fatty acids, or else a dilution by adding oils with a different lipid profile.

“Lipid composition” means a mixture of lipids comprising at least one PUFA-rich oil according to the invention, extracted from crude, refined and/or modified biomass.

Unless otherwise indicated, the percentages are given by mass.

### II. Biomass Cultivation

The present invention relates to a method for preparing a biomass of protists of the genus Pythium comprising PUFA-rich lipids, said method comprising (a) the cultivation of protists of the genus Pythium on a cultivation medium comprising a carbon source, and (b) the recovery of the biomass from the cultivation medium, the cultivation step comprising an illumination phase.

The cultivation of protists of the genus Pythium in heterotrophy on a cultivation medium comprising a carbon source is well known to the person skilled in the art. These cultivations can be in discontinuous mode called “batch”, in semi-continuous mode called “fed batch” or in continuous mode.

The method according to the invention is advantageously implemented for an industrial production of biomass.

Whether in discontinuous, semi-continuous or continuous mode, the cultivation step (a) consists in producing a fermentation broth comprising the fermentation juice and the biomass, with a density of at least 20 g/L of dried matter. Advantageously, the cultivation will be carried out so as to reach a density of at least 30 g/L, advantageously of at least 40 g/L.

Once the desired cultivation density has been reached, the biomass is recovered from the cultivation medium, separated from the fermentation juice.

The cultivation of protists of the genus Pythium is well known to the person skilled in the art who will know how to determine the conditions for implementing these cultivations in aerobic mode to obtain the desired density. Mention will be made in particular of the cultivation methods described in Stinson and al. (1991), US2014256973, which the person skilled in the art can adapt to an industrial cultivation.

The inocula used for the cultivation of Pythium can be obtained by two methods.

The first one consists in performing a cultivation on agar favorable to the production of spores, for example the PDA (Potato Dextrose Agar) or Czapek Dox agar medium. After an adapted growth time, the spores are harvested in a liquid medium according to a method known to the person skilled in the art. For example, water or an adapted saline medium is used to flood the aerial part of the mycelium which has grown on the agar. The spore-laden liquid is then recovered and the spores counted.

Inoculation takes place in a liquid cultivation in a baffled Erlenmeyer type flask containing a medium suitable for growth such as PDB (Potato Dextrose Broth) or Czapek Dox. From 100 to 10000 spores per milliliter of medium are added to the cultivation medium. Inoculation with an adapted number of spores allows to limit the growth in the form of balls which is a characteristic of the growth of filamentous microorganisms.

A second method consists in recovering wet biomass from a cultivation in a flask then homogenizing this biomass. This biomass can be in the form of balls. Homogenization is carried out in a device such as a blender or other homogenizer. 5 to 15% of this homogenate is used to inoculate a cultivation in a baffled Erlenmeyer type flask and in a medium suitable for growth such as PDB (Potato Dextrose Broth) or Czapek Dox. Homogenization allows to limit the formation of balls during the cultivation thus inoculated.

Growth is carried out in a shaking incubator at a temperature generally ranging from 20 to 30° C. The orbital shaking is set between 100 and 200 revolutions per minute. After a growth time of the order of 5 to 15 days, generally 7 days, the biomass can be recovered to inoculate another cultivation of a larger volume, such as a fermenter, or be harvested or analyzed.

The invention consists in illuminating the cultivation considering all or part of the cultivation step (mixotrophy).

Usually, the cultivation of Pythium is carried out in heterotrophy, that is to say without a light source, since this microorganism is not photosynthetic and therefore does not need it for its growth. Such cultivations under heterotrophic conditions are described in particular in the references mentioned above and in WO 2009/143007, by Cheng & al (1999) or by Stinson & al (1991). The inventors unexpectedly observed that the illumination of the cultivation allowed to increase the lipid content in the cells of protists of the genus Pythium compared to a heterotrophic cultivation described in the state of the art.

This illumination consists in illuminating the protists in the cultivation medium in a controlled manner at an illumination intensity comprised between 50 and 2000 µmoles of photons/m²/sec. In this sense, it is indeed a particular illumination, generally by appropriate illumination means to illuminate the cultivation medium and motivated to favor the production of PUFA which is distinguished from a possible indirect illumination which would come from the light used to illuminate the place where the cultivation step is implemented, such as a laboratory, whether natural or artificial.

The wavelengths adapted for illuminating Pythium cultivations according to the invention are preferably in a spectrum which goes from UV to near infrared via the visible, generally comprised between 300 nm and 750 nm. This may be a light called “white” light, the radiation spectrum of which generally ranges from 400 to 750 nm, Ultra-Violet (UV) radiation, the radiation spectrum of which is comprised between 300 and 430 nm, a light called “blue” light, the radiation spectrum of which generally ranges from 430 to 500 nm, a light called “green” light, the radiation spectrum of which generally ranges from 500 to 570 nm, a light called “yellow” light, the radiation spectrum of which generally ranges from 570 to 590 nm, or else a light called “red” light, the radiation spectrum of which generally ranges from 590 to 750 nm.

The light intensity can be comprised between 20 and 5000 µmoles/photons/seconds, preferentially between 50 and 3000 µmoles/photons/seconds, more preferentially between 50 and 2000 µmoles/photons/seconds. In particular, the light intensity can be at least 200 µmoles/photons/seconds, from 200 to 1000 µmoles/photons/seconds, in particular about 500 µmoles/photons/seconds.

The light intensity used for a light illumination called “white” light illumination is advantageously comprised between 100 and 1000 µmoles of photons/m²/sec.

The light intensity used for UV illumination is advantageously comprised between 50 and 500 µmoles of photons/m²/sec.

The light intensity used for blue light illumination is advantageously comprised between 50 and 500 µmoles of photons/m²/sec.

The light intensity used for green light illumination is advantageously comprised between 50 and 500 µmoles of photons/m²/sec.

The light intensity used for yellow light illumination is advantageously comprised between 100 and 1000 µmoles of photons/m²/sec.

The light intensity used for red light illumination is advantageously comprised between 50 and 1000 µmoles of photons/m²/sec.

The composition of the light spectrum will depend in particular on the lamps used for culture illumination. These are in particular lamps of the fluorescent or neon tube type, of the sodium lamp type or else of the LED type, preferably of the LED type.

For light illumination called “white” light illumination, LED-type lamps will advantageously be used, the emission spectrum of which is comprised between 400 and 750 nm. These white LED-type sources generally have a main emission peak in the blue around 470 nm and a more diffuse secondary emission between 500 and 700 nm.

For light illumination called “UV” light illumination, LED-type lamps will advantageously be used, the emission spectrum of which is comprised between 380 and 400 nm.

For light illumination called “blue” light illumination, LED-type lamps will advantageously be used, the emission spectrum of which is comprised between 450 and 500 nm.

For light illumination called “green” light illumination, LED-type lamps will advantageously be used, the emission spectrum of which is comprised between 500 and 570 nm.

For light illumination called “yellow” light illumination, LED-type lamps will advantageously be used, the emission spectrum of which is comprised between 570 and 590 nm.

For light illumination called “red” light illumination, LED-type lamps will advantageously be used, the emission spectrum of which is comprised between 590 and 750 nm.

The illumination can be implemented throughout the duration of the cultivation step (a), or partially. The person skilled in the art will be able to determine when to start the illumination and when to stop it at the end of the cultivation before step (b) of recovering the biomass.

The duration of the illumination can range from 10 to 100% of the time of implementation of the cultivation step (a), preferentially between 30 and 100%, more preferentially between 50 and 100%.

Partial illumination can be provided during a phase conducive to the accumulation of lipids, generally from the middle or the end of the exponential phase of growth.

The illumination may be continuous, or discontinuous with alternating periods of light and periods of darkness. The successive illumination phases can be comprised between 1 second and 10 minutes, for example between 10 seconds and 2 minutes, or else between 20 seconds and 1 minute. They are spaced out by dark phases of a duration equal to or different from the duration of each illumination phase.

Industrial mixotrophic cultivation devices for varying illumination, both in wavelength, intensity and duration, are for example described in patent applications WO 2009/069967, US 2010/005711, WO 2014/ 174182 or WO 2019/034792.

When the light intensity to be delivered is chosen at a high value, in particular beyond 500 µmoles of photons/m²/sec, the electrical consumption and the release of heat can be limited by supplying the light in the form of a high-frequency flash, generally comprised between 0.5 and 150 kHz, advantageously between 1 and 100 kHz, which results in an alternation of lit phases and dark phases in a period comprised between 0.001 and 0.00001 seconds. The duration of the lit phase can be from 1 to 90% of each period, depending on the reductions in electrical consumption and heat release desired. The use of electrical equipment and the configuration thereof are known to the person skilled in the art.

According to a preferred embodiment of the invention, the illumination will be provided by a light source of the LED type delivering red illumination, the emission spectrum of which will be comprised between 600 and 750 nm and the duration of which will be comprised between 50 and 100% of the cultivation step.

### III. Biomass Recovery

Step (b) of recovering the biomass from the cultivation medium comprises the separation of the biomass from the fermentation juice.

The recovery methods are well known to the person skilled in the art, in particular by centrifugation (plate centrifuge or sedicanter), or by filtration (plate filter, press filter, ceramic or organic cross-flow filtration).

The raw biomass recovered can be washed to eliminate certain solubles (for example by filtration to eliminate the fermentation medium and washing with water).

The raw or washed biomass can also be dried by usual methods, in particular by atomization or freeze-dried.

It can also be treated by adding components useful for its preservation and storage, such as antioxidants, before or after drying, or sugars to prevent spontaneous heating of the biomass.

The invention also relates to a biomass of protists of the genus Pythium comprising PUFA-rich lipids, the lipid content being at least 30% relative to the dry matter (% DM), preferably at least 50% DM, more preferentially, of at least 55% DM, in particular at least 56, 57, 58, 59, 60, 61, 62, 63, 64 or 65% DM, up to 75% DM.

PUFAs are essentially made up of a mixture of ARA and EPA. The ARA/EPA weight ratio is advantageously from 0.5 to 1, more particularly from 0.6 to 0.9, in particular approximately 0.7.

The PUFA (ARA + EPA) content is at least 10% of the sum of the fatty acids, advantageously at least 15%, in particular from 15 to 40%. According to a particular embodiment, the total content of ARA and EPA is at least 20%.

The ARA content is at least 5%, preferably at least 6%, more preferably 7.5% or more than 7.5%, in particular at least 7.7%, at least 8% or at least 9%.

The EPA content is preferably at least 12% of the sum of fatty acids, more preferably at least 13%, in particular at least 14%, at least 15%, at least 16% or at least 17 %.

The fatty acid content in the crude or refined oils according to the invention is advantageously as follows:

| | |
|---|---|
| myristic acid (C14:0) | 5% to 10% |
| palmitic acid (C16:0) | 10% to 20% |
| palmitoleic acid (C16:1 n-7) | 6% to 10% |
| oleic acid (C18:1 n-9c) | 15% to 25% |
| linoleic acid (C18:2 n-6c) | 10% to 25% |
| arachidonic acid (C20:4 n-6) | 5% to 15% |
| eicosapentaemoic acid (C20:5 n-3) | 7% to 20% |

More advantageously, the fatty acid content in the crude or refined oils according to the invention is as follows:

| | |
|---|---|
| myristic acid | (C14:0) 6% to 7% |
| palmitic acid (C16:0) | 11% to 16% |
| palmitoleic acid (C16:1 n-7) | 6% to 8.5% |
| oleic acid (C18:1 n-9c) | 16% to 25% |
| linoleic acid (C18:2 n-6c) | 13% to 22% |
| arachidonic acid (C20:4 n-6) | 6% to 10% |
| eicosapentaemoic acid (C20:5 n-3) | 10% to 20% |

In particular, the content of the main fatty acids is around 9% for myristic acid (C14:0); 12% for palmitic acid (C16:0); 7% for palmitoleic acid (C16:1 n-7); 17% for oleic acid (C18:1 n-9c); 20% for linoleic acid (C18:2 n-6c); 9% for arachidonic acid or ARA (C20:4 n-6) and 18% for eicosapentaemoic acid or EPA (C20:5 n-3).

According to a particular embodiment, the biomass according to the invention has been “stabilized” for being preserved stored and/or transported.

This may involve the addition of stabilizing components that are not associated with protist cells in nature, such as sugars (WO 2020/036814), antioxidants such as tocopherol, rosemary extract or ascorbic acid.

According to a particular embodiment, the biomass is no longer able to develop on a cultivation medium, the vital functions of the cells being altered. This is particularly the case for degraded biomass, in particular degraded and dry biomass.

### IV. Extraction of Lipids

The invention also relates to a method for preparing lipid compositions rich in PUFA which comprises the cultivation from a) the cultivation of protists of the genus Pythium on a cultivation medium comprising a carbon source, (b) the recovery of the biomass from the cultivation medium and (c) the extraction of the PUFA-rich lipids from the recovered biomass, the cultivation step (a) comprising an illumination phase.

Several industrial methods for extracting PUFA-rich lipids from a biomass of microalgae cultivated to produce said oils are described in the literature and known to the person skilled in the art. Lipid extraction involves lysing biomass cells to release the PUFA-rich oils they contain, then separating lipids from solid fractions and water-soluble fractions.

Lysis can be mechanical (pressing or grinding) or enzymatic with proteases or cellulases.

Mechanical lysis methods are well known, in particular by ball mill, mixer-disperser, high pressure homogenizer, pin mill or impact mill, ultrasound, pulsed electric fields. As devices for the implementation of these mechanical lysis methods, particular mention will be made (name of the manufacturer in parentheses) for the ball mill: Discus-1000 (Netzsch); ECM-AP60 (WAB); for the high pressure homogenizer: Ariete (GEA); for the mixer-disperser: 700-X (Silverson), for the pin mill: Contraplex (Hosakawa); for the impact mill: Condux (Netzsch).

The enzymes capable of being used are known, in particular described in WO2015/095688, WO2011/153246, US6750048 and WO2015/095694, in particular proteases or cellulases such as the enzymes marketed by the company Novozyme under the names Alcalase 2.5 L, Alcalase 2.4 L, Alcalase 3.0 T, Novozym 37071, Flavourzyme 1000 L, Novozym FM 2.4 L, Protamex, Viscozyme. The conditions of implementation are those recommended by the supplier, the temperature being that recommended for optimal activity of the enzymes, at least 50° C. and up to 70° C., preferably around 65° C. Advantageously, the enzymatic lysis is carried out under an oxygen-poor atmosphere. Generally, the oxygen concentration is less than 1% by mass.

Mention will be made in particular of the lysis methods described in application WO 2020/053375, WO 2001/053512.

The extraction of oils from lysed biomass can be done by various known methods, such as the addition of sodium in the form of sodium sulfate or sodium chloride (WO 2011/153246), solvent extraction (US 2014/350222) and/or high temperatures for several hours (WO 2015/095694), or by many other methods described in the literature (WO 2019/219396, WO 2019/219443, WO 2019/121752, WO 2018 /122057, WO 2018/013670, WO 2018/011286, WO 2018/011275, WO 2018/013670, WO 2018/011286, WO 2018/011275, WO 2015/095696, WO 2011/153246, WO 2002/010423).

Preferably, the extraction of oils from lysed biomass is carried out by mechanical separation, also well known to the person skilled in the art, such as gravity separation, in particular by centrifugation as described in patent application WO 01/53512. Use can also be made of a continuous separation, in particular by centrifugal separator with plates. Such separators are known for continuously extracting oils from complex media comprising solid residues and water, as described in patent application WO 2010/096002, in particular marketed by the companies Alfa Laval, Flottweg or GEA Westfalia, in particular. This continuous separation step is preferred in the method used to obtain the oil according to the invention.

In some cases, the lysis and extraction steps are simultaneous, under a mechanical action, generally by one or more centrifugations (WO 2019/032880) in particular with the centrifugation devices described above.

The oil extraction from biomass may favor the extraction of these PUFAs over lower molecular weight saturated fatty acids. Advantageously, this concentration does not substantially modify the intrinsic properties of the oil contained in the biomass, in particular the triglyceride content. Preferably, the oil according to the invention is an oil which has not undergone substantial modifications in its fatty acid content by the addition of PUFA, for example in the form of esters, by concentration and/or by elimination of saturated fatty acids such as palmitic acid.

The oil obtained is generally an oil called crude oil, which can be used as it is or be refined, in particular to facilitate its preservation, by preventing it from going rancid, or to modify its color or its smell so as to make it more acceptable to a consumer. These purification and refining steps are well known to the person skilled in the art, described in patent applications (WO 2002/010322, WO 2017/035403), in particular the steps of degumming, neutralizing the free fatty acids, of decoloration and deodorization. They eliminate (all or in part) phospholipids, pigments, volatiles and free fatty acids. In fact, these methods do not substantially modify the relative content of fatty acids, saturated or unsaturated, nor the triglyceride content of the refined oil obtained compared to the purified oil.

The method according to the invention can also comprise a step of modifying the oils obtained previously and described above, whether they are crude, purified or refined.

Some methods of the state of the art have a step called “winterization” step implemented on crude or purified oils, in particular to remove saturated fatty acids, with the effect of increasing the PUFA content (WO 02/10322). The crude or refined oils extracted according to the invention do not a priori require “winterization” to be exploited. However, the person skilled in the art may choose to add such a “winterization” step if they find any commercial advantage therein for their final product.

Certain methods comprise a modification of the oils recovered from the biomass, for example to modify the composition thereof in terms of certain saturated or unsaturated fatty acids, in particular to promote the concentration of PUFA. Such methods known to the person skilled in the art comprise in particular enzymatic treatments with enzymes such as lipases (CN 105349587, WO 2019/219904, WO 2019/219903).

Other components can be added to the crude, purified or refined oils according to the invention, such as antioxidants, in particular those described in applications WO 2019/185942, WO 2019/185940, WO 2019/185939, WO 2019/185910, WO 2019/185894, WO 2019/185889 and WO 2019/185888.

Crude or refined oils, or oils of modified composition can also be diluted for their subsequent use. The oils used to dilute the PUFA-rich oil obtained by the method according to the invention are generally and preferably vegetable oils suitable for human or animal food consumption. Mention will be made in particular of sunflower, rapeseed, soya, walnut, sesame, hemp, hazelnut, argan, olive, linseed oils, or any other oil suitable for food use. The added oil can also be an oil comprising other PUFAs, in particular DHA, in particular other oils of microbial origin or else fish oils.

The method according to the invention may further comprise such a step of diluting a crude or refined oil obtained previously.

The invention also relates to a PUFA-rich oil obtained by the method according to the invention, which is crude or refined or capable of being obtained by the method according to the invention.

The composition of the oil according to the invention, in particular the content of PUFA and of other fatty acids, is given above for the composition of the oil contained in the biomass.

### V. Compositions

The invention also relates to a method for preparing a pharmaceutical, cosmetic, nutraceutical or food composition comprising a biomass of protists of the genus Pythium comprising PUFA-rich lipids, said method comprising (a) the cultivation of protists of the genus Pythium on a cultivation medium comprising a carbon source, (b) the recovery of the biomass from the cultivation medium and (d) the formulation of a composition by adding the biomass recovered in (b) to usual components of the pharmaceutical, cosmetic, nutraceutical or food compositions, the cultivation step (a) comprising an illumination phase as defined above.

The invention also relates to a method for preparing a pharmaceutical, cosmetic, nutraceutical or food composition comprising a lipid composition rich in PUFA, said method comprising (a) the cultivation of protists of the genus Pythium on a cultivation medium comprising a carbon source, (b) the recovery of biomass from the cultivation medium, (c) the extraction of the PUFA-rich lipids from the recovered biomass, and (d) the formulation of a composition by adding the lipid composition extracted in (c) to usual components of the pharmaceutical, cosmetic, nutraceutical or food compositions, the cultivation step (a) comprising an illumination phase as defined above.

The invention also relates to a composition which comprises a biomass or a PUFA-rich oil that can be obtained by the method according to the invention as described above.

A composition according to the invention may comprise one or more excipients. An excipient is a component, or mixture of components, which is used in the present invention to impart desirable characteristics to the composition for its preservation and use, including food, pharmaceutical, cosmetic and industrial compositions. An excipient can be described as a “pharmaceutically acceptable” excipient when it is added to a pharmaceutical composition whose properties are known from the pharmacopoeia to be used in contact with human and animal tissues without excessive toxicity, irritation, allergic reaction or other complications. Different excipients can be used like organic or inorganic base, organic or inorganic acid, pH buffer, stabilizer, antioxidant, adhesion agent, parting agent, coating agent, outer phase component, a controlled release component, a surfactant, a humectant, a filler, an emollient or combinations thereof.

Depending on their destination, the compositions according to the invention are in particular pharmaceutical, cosmetic, nutraceutical or food compositions.

Foods are intended for both humans and animals and comprise solid, paste or liquid compositions. In particular, mention may be made of common foods, liquid products, including milks, drinks, therapeutic drinks and nutritional drinks, functional foods, supplements, nutraceuticals, infant formulas, including formulas for premature infants, foods for pregnant or breastfeeding women, food for adults, geriatric food and animal feed.

The PUFA-rich oil obtained by the method according to the invention, whether crude or refined, or the biomass which contains it, can be used directly as or added as an additive in an oil, a spread, another fatty ingredient, a drink, a soy-based sauce, dairy products (milk, yogurt, cheese, ice cream), bakery products, nutritional products, for example in the form of a nutritional supplement (in capsule or tablet form), vitamin supplements, food supplements, powders to be diluted for drinks, such as energy drinks or milk powders for infant formulas, finished or semi-finished powdered food products, etc., according to the practices known to the person skilled in the art.

Animal feeds are also known to the person skilled in the art. They are particularly intended for livestock, such as cows, pigs, chickens, sheep, goats or in fish farming for crustaceans or farmed fish.

Pharmaceutical compositions comprising a PUFA-rich oil are also known to the person skilled in the art, the oil being used alone or in combination with other drugs.

The PUFA-rich oil obtained by the method according to the invention, which is crude or refined, or the biomass which contains it, can be formulated in the form of single-dose compositions, in particular in the form of tablets, capsules, powders, granules, adapted for oral administration.

The invention also relates to the use of a PUFA-rich oil obtained by the method according to the invention, which is crude, refined or diluted, for human or animal food, in particular for the food of newborns, children, or pregnant or breastfeeding women.

Such uses are well known to the person skilled in the art, described in particular in patent application WO 2010/107415 and on the website of the company DSM (https://www.dsm.com/markets/foodandbeverages/en_US/ products/nutritional-lipids/life-dha.html)

### EXAMPLES

### Example 1

The Pythium irregulare strain from NBRC collection n°30346 is cultivated in a 250 mL baffled Erlenmeyer flask in 50 mL of PDB (Potato Dextrose Broth) cultivation medium composed of 200 g of potato infusion and 20 g of dextrose. The wet biomass is recovered by centrifugation at 10000 g for 5 minutes then homogenized by vortexing in the presence of glass beads with a diameter of 1 mm and an amount representing a volume of 1 to 2 mL. 5 mL of this homogenate is used to inoculate a 250 mL flask containing 45 mL of PDB medium. The flasks are incubated at a temperature of 26° C. and with magnetic stirring by a bar magnet at 150 revolutions per minute. The light is provided from below via a device comprising several LEDs of an identical nature, continuously throughout the duration of the cultivation step. Each of the cultivation stations by magnetic agitation can be equipped with an illumination device of a different nature. The intensity of the illumination is fixed at 200 µmoles of photons/m²/sec. An indicator of growth in heterotrophy is performed under the same experimental conditions except for the light which is absent. The cultivations are incubated under these conditions for 7 days. The biomass is harvested by centrifugation at 10000 g for 5 minutes and the supernatant discarded. After washing the biomass with distilled water, it is freeze-dried. The lipid content of the biomass is analyzed by GC-FID after a transesterification step on an aliquot of approximately 2 mg of this freeze-dried biomass.

The results of the lipid analysis are shown in Table 1. The percentages of fatty acids are given in relation to the total mass of fatty acid

| | Indicator | UV 385 nm | Blue 455 nm | Green 521 nm | Yellow | Red 593 nm | Red 660 nm | White |
|---|---|---|---|---|---|---|---|---|
| FM/DM% | 23.6 | 61.6 | 60.2 | 43.3 | 52.3 | 36.7 | 68.10 | 30.3 |
| C14:0 | 6.1 | 7.1 | 7.0 | 6.7 | 6.3 | 6.9 | 8.5 | 6.6 |
| C16:0 | 14.6 | 14.3 | 13.0 | 14.3 | 16.1 | 13.2 | 15.2 | 13.4 |
| C16:1 | 5.7 | 8.2 | 7.7 | 7.0 | 8.6 | 7.9 | 7.1 | 6.4 |
| C18:1 | 15.9 | 22.3 | 19.9 | 21.2 | 23.7 | 21.0 | 23.2 | 19.0 |
| C18:2 | 18.2 | 15.2 | 14.7 | 13.5 | 14.8 | 13.5 | 15.3 | 15.0 |
| ARA | 7.0 | 6.7 | 7.5 | 6.4 | 5.5 | 6.4 | 7.7 | 7.8 |
| EPA | 11.7 | 10.8 | 11.8 | 13.8 | 8.1 | 12.5 | 10.9 | 12.7 |

Depending on the light used, with the cultivation method according to the invention compared to a cultivation in heterotrophy (without light) of the state of the art a production of ARA compared to the dry matter multiplied by a factor of at least 1.5, on average by a factor of about 2.4, up to a factor of about 3.7 is observed.

EPA production relative to dry matter is multiplied by a factor of at least 2.5, on average by a factor of about 3.6, up to a factor of about 4.7.

The total production of ARA and EPA relative to the dry matter is multiplied by a factor of at least 2.1, on average by a factor of about 3.1, up to a factor of about 4.2.

### Example 2

The Pythium irregulare strain from NBRC collection n°100109 is cultivated in a 250 mL baffled Erlenmeyer flask in 50 mL of PDB (Potato Dextrose Broth) cultivation medium composed of 200 g of potato infusion and 20 g of dextrose. The wet biomass is recovered by centrifugation at 10000 g for 5 minutes then homogenized by vortexing in the presence of glass beads with a diameter of about 1 mm and an amount representing a volume of 1 to 2 mL. 5 mL of this homogenate is used to inoculate a 250 mL flask containing 45 mL of PDB medium. The flasks are incubated at a temperature of 26° C. and with magnetic stirring by a bar magnet at 150 revolutions per minute. The light is provided from below via a device comprising several LEDs having an emission peak at 660 nm, continuously throughout the duration of the cultivation step. The intensity of the illumination is fixed at 200, 500 or 2000 µmoles of photons/m²/sec. A reference of growth in heterotrophy is performed under the same experimental conditions except for the light which is absent. The cultivations are incubated under these conditions for 7 days. The biomass is harvested after 7 days by centrifugation at 10000 g for 5 minutes and the supernatant discarded. After washing the biomass with distilled water, it is freeze-dried. The lipid content of the biomass is analyzed by GC-FID after a transesterification step on an aliquot of approximately 2 mg of this freeze-dried biomass.

The results of the lipid analysis are shown in Table 2.

The percentages of fatty acids are given in relation to the total mass of fatty acid

| | Indicator | 200 µE | 500 µE | 2000 µE |
|---|---|---|---|---|
| FM/DM% | 19.1 | 61.5 | 73.3 | 66.9 |
| C14:0 | 9.5 | 8.7 | 8.8 | 9.2 |
| C16:0 | 18.1 | 12.8 | 12.5 | 11.3 |
| C16:1 | 12.0 | 7.2 | 8.1 | 6.7 |
| C18:1 | 20.8 | 19.6 | 19.3 | 16.9 |
| C18:2 | 18.6 | 19.4 | 18.2 | 21.0 |
| ARA | 7.5 | 9.2 | 9.3 | 9.9 |
| EPA | 8.2 | 14.7 | 13.7 | 17.6 |

According to the light intensity, with the cultivation method according to the invention compared to a cultivation in heterotrophy (without light) of the state of the art a production of ARA compared to the dry matter multiplied by a factor of at least 3.4, on average by a factor of about 3.9, up to a factor of about 4 is observed.

EPA production relative to dry matter is multiplied by a factor of at least 3.3, on average by a factor of about 3.7, up to a factor of about 4.3.

The total production of ARA and EPA relative to the dry matter is multiplied by a factor of at least 3.3, on average by a factor of about 3.8, up to a factor of about 4.2.

### Example 3

The Pythium irregulare strain from NBRC collection n°100109 is cultivated in a 250 mL baffled Erlenmeyer flask in 50 mL of PDB (Potato Dextrose Broth) cultivation medium composed of 200 g of potato infusion and 20 g of dextrose. The wet biomass is recovered by centrifugation at 10000 g for 5 minutes then homogenized by vortexing in the presence of glass beads with a diameter of about 1 mm and an amount representing a volume of 1 to 2 mL. 5 mL of this homogenate is used to inoculate a 250 mL flask containing 45 mL of PDB medium. The flasks are incubated at a temperature of 26° C. in an incubator whose stirring is set at 140 revolutions per minute. The light is provided by a series of fluorescent tubes of the daylight type at 6000°K. The intensity of the illumination is of the order of 50 µmoles of photons/m²/sec. The cultivations are incubated under these conditions for 7 days. The biomass is harvested after 7 days by centrifugation at 10000 g for 5 minutes and the supernatant discarded. After washing the biomass with distilled water, it is freeze-dried. The lipid content of the biomass is analyzed by GC-FID after a transesterification step on an aliquot of approximately 2 mg of this freeze-dried biomass.

The results of the lipid analysis are shown in Table 3.

The percentages of fatty acids are given in relation to the total mass of fatty acid.

| | A | B | AVERAGE HETEROTROPHY |
|---|---|---|---|
| FM/DM% | 45.8 | 47.1 | 21.3 |
| C14:0 | 9.0 | 8.6 | 7.8 |
| C16:0 | 16.2 | 15.5 | 16.4 |
| C16:1 | 16.9 | 14.7 | 8.9 |
| C18:1 | 21.4 | 20.1 | 18.8 |
| C18:2 | 15.8 | 16.5 | 18.4 |
| ARA | 4.1 | 8.8 | 7.2 |
| EPA | 5.8 | 5.7 | 10.0 |

A marked increase in the lipid content in the dry biomass is observed with the cultivation method according to the invention, compared with cultivations in heterotrophy (without light). An increase of more than 60% in PUFA in comparison with the values generally observed in heterotrophic cultivation is observed, with respect to the dry matter.

### REFERENCES

Athalye SK, Garcia RA, Wen Z., Use of biodiesel-derived crude glycerol for producing eicosapentaenoic acid (EPA) by the fungus Pythium irregulare, J Agric Food Chem. 2009 Apr 8;57(7):2739-44. doi: 10.1021/jf803922w.

Gandhi SR, Weete JD., Production of the polyunsaturated fatty acids arachidonic acid and eicosapentaenoic acid by the fungus Pythium ultimum, J Gen Microbiol. 1991 Aug;137(8):1825-30.

Liang Y, Zhao X, Strait M, Wen Z., Use of dry-milling derived thin stillage for producing eicosapentaenoic acid (EPA) by the fungus Pythium irregulare, Bioresour Technol. 2012 May;111:404-9. doi: 10.1016/j.biortech.2012.02.035.

Ren, Liang; Zhou, Pengpeng; Zhu, Yuanmin; Zhang, Ruijiao; Yu, Lo -- Improved eicosapentaenoic acid prod; Applied Microbiology and Biotechnology Volume 101 issue 9 2017 [doi 10.1007_s00253-016-8044-0]

Lio J, Wang T., Pythium irregulare fermentation to produce arachidonic acid (ARA) and eicosapentaenoic acid (EPA) using soybean processing co-products as substrates, Appl Biochem Biotechnol. 2013 Jan;169(2):595-611. doi: 10.1007/s12010-012-0032-y.

Manjula S, Subramanian R. Membrane technology in degumming, dewaxing, deacidifying, and decolorizing edible oils. Crit Rev Food Sci Nutr. 2006;46(7):569-92. Review. PubMed PMID: 16954065.

Stinson EE, Kwoczak R, Kurantz MJ., Effect of cultural conditions on production of eicosapentaenoic acid by Pythium irregulare. Journal of Industrial Microbiology & Biotechnology Volume 8 issue 3 1991 [doi 10.1007_bf01575850]

Wu L, Roe CL, Wen Z., The safety assessment of Pythium irregulare as a producer of biomass and eicosapentaenoic acid for use in dietary supplements and food ingredients, Appl Microbiol Biotechnol. 2013 Sep;97(17):7579-85. doi: 10.1007/s00253-013-5114-

CN 105349587, GB 2 031 290, US 4,971,660, US 5,310,487, US 6,750,048, US 2010/005711, US 2014/256973, US 2014/350222, WO 2001/053512, WO 2002/010423, WO 2002/10322, WO 2009/069967, WO 2009/143007, WO 2010/096002, WO 2010/107415, WO2011/153246, WO 2014/174182, WO 2015/095688, WO2015/095694, WO 2015/095696, WO 2017/035403, WO 2018/011275, WO 2018/011286, WO 2018/013670, WO 2018/122057, WO 2019/034792, WO 2019/032880, WO 2019/121752, WO 2019/185888, WO 2019/185889, WO 2019/185894, WO 2019/185910, WO 2019/185939, WO 2019/185940, WO 2019/185942, WO 2019/219903, WO 2019/219904, WO 2019/219396, 2019/219443, WO 2020/053375

Cheng M H & al “Fungal production of eicosapentaenoic and arachidonic acids from industrial waste streams and crude soybean oil”; Bioresource technology, Elsevier, Amsterdam, NL: vol. 67, no. 2, Jan. 1, 1999 (1999-01-01), pages 101-110, XP001016139

Stinson E E & al “Effect of cultura conditions on production of eicosapentaenoic acid by Pythium-irregulare”; Journal for industrial microbiology, Society for industrial Microbiology, UK, vol. 8, no. 3, Jan. 1, 1991 (1991-01-01), pages 171-178, XP008085449

1. A method for preparing a biomass of protists of the genus Pythium comprising PUFA-rich lipids, said method comprising
(a) the cultivation of protists of the genus Pythium on a cultivation medium comprising a carbon source, and
(b) the recovery of the biomass from the cultivation medium,
wherein the cultivation step comprises an illumination phase, said phase consisting in illuminating the protists in the cultivation medium in a controlled manner at an illumination intensity comprised between 50 and 2000 µmoles of photons/m²/sec.

2. The method according to claim 1, wherein the protists of the genus Pythium are protists of the species Pythium irregulare.

3. The method according claim 1, wherein the illumination phase is implemented during all or part of the cultivation step (a).

4. The method according to claim 3, wherein the duration of the illumination ranges from 10 to 100% of the time of implementation of the cultivation step (a).

5. The method according to claim 3, wherein the duration of the illumination ranges from 50 to 100% of the time of implementation of the cultivation step (a).

6. The method according to claim 1, wherein the illumination intensity is comprised between 100 and 1000 µmoles of photons/m²/sec.

7. The method according to claim 1, wherein the illumination phase is implemented by light illumination, selected among white light illumination, blue light illumination, green light illumination, yellow light illumination and red light illumination.

8. The method according to claim 1, wherein the illumination phase is implemented by UV light illumination.

9-12. (canceled)

13. The method for preparing PUFAs from a biomass of protists of the genus Pythium comprising PUFA-rich lipids, said method comprising
(a) the cultivation of protists of the genus Pythium on a cultivation medium comprising a carbon source,
(b) the recovery of the biomass from the cultivation medium, and
(c) the extraction of the PUFA-rich lipids from the biomass recovered in (b),
wherein the cultivation step a) comprises an illumination phase, said phase consisting in illuminating the protists in the cultivation medium in a controlled manner at an illumination intensity comprised between 50 and 2000 µmoles of photons/m²/sec.

14. The method according to claim 13, wherein the protists of the genus Pythium are protists of the species Pythium irregulare.

15. The method according to claims 13, wherein the illumination phase is implemented during all or part of the cultivation step (a).

16. The method according to claim 15, wherein the duration of the illumination ranges from 10 to 100% of the time of implementation of the cultivation step (a).

17. The method according to claim 15, wherein the duration of the illumination ranges from 50 to 100% of the time of implementation of the cultivation step (a).

18. The method according to claim 13, wherein the illumination intensity is comprised between 100 and 1000 µmoles of photons/m²/sec.

19. The method according to claim 13, wherein the illumination phase is implemented by light illumination, selected among white light illumination, blue light illumination, green light illumination, yellow light illumination and red light illumination.

20. The method according to claims 13 to 18, wherein the illumination phase is implemented by UV light illumination.

21-28. (canceled)
